(19) United States
(12) Patent Application Publication
(10) Pub. No.: US 2016/0367147 A1
(43) Pub. Date: Dec. 22, 2016

(54) SELF-INSPECTION DEVICE

(71) Applicant: Wilbert L. Watson, Chesterfield, VA (US)

(72) Inventor: Wilbert L. Watson, Chesterfield, VA (US)

(21) Appl. No.: 14/741,979

(22) Filed: Jun. 17, 2015

(51) Int. Cl. A61B 5/00; G02B 7/182

(52) U.S. Cl. CPC A61B 5/0079; G02B 7/182

(57) ABSTRACT

A self-inspection device which includes: a belt; an elbow attachment, where said elbow attachment is positioned at the center of the belt; a telescoping post extending at a vertical angle from the elbow connector; and a mirror, wherein said mirror is attached to the telescoping post. The self-inspection device may further include a pivoting connector, where pivoting connector connects the mirror to the telescoping post. The present invention also relates to a method for self inspection of the genital and rectum areas of a user comprising: wrapping a belt around a waist of a user; positioning a mirror between the legs of the user; and pivoting the mirror to view the genital and rectum areas of the user.

### BACKGROUND OF THE INVENTION

Field of Invention

The present invention relates to a device used to provide self-examination of the rectum and genital areas of an individual.

Description of Related Art

On certain occasions it may become necessary for an individual to perform a self-inspection of their genital and rectal areas for personal hygiene or medical purposes. As is well known it is difficult to view these areas due to the general anatomy of most individuals. However it is important to view these areas due to hygiene or medical purposes. The prior art includes certain devices that have been developed for self-inspection such as U.S. Pat. No. 5,311,366 to Gerace. This particular device disclosed in the Gerace shows a personal hygiene mirror assembly that is mountable on an individual's legs while in a sitting position which includes grips for each leg and a mirror that is positioned at the center of the device. One drawback to this particular device is one must be in a seated position and the angles of the device may be limited due to the use in a seated position.

As a result, it would be advantageous to have such a self-examination device so that it could be used in a standing position without the engagement of leg grips thus requiring the individual to be seated or in a reclined position.

### SUMMARY OF THE INVENTION

The present invention relates to a self-inspection device which includes: a belt; an elbow attachment, where said elbow attachment is positioned at the center of the belt; a telescoping post extending at a vertical angle from the elbow connector; and a mirror, wherein said mirror is attached to the telescoping post. The self-inspection device may further include a pivoting connector, where pivoting connector connects the mirror to the telescoping post. The present invention also relates to a method for self inspection of the genital and rectum areas of a user comprising: wrapping a belt around a waist of a user; positioning a mirror between the legs of the user; and pivoting the mirror to view the genital and rectum areas of the user.

### BRIEF DESCRIPTION OF DRAWINGS

FIG. 1 depicts a self-examination device in accordance with the present invention.

### DETAILED DESCRIPTION

The present invention relates to a self-examination device that is used to provide a means for self-examination of the rectum and genital areas of an individual. The present invention may be used to inspect these areas through the use of a mirror while in a standing position. Preferably, the self-examination device includes a belt that is worn around the waist of the individual and a telescoping post that extends at the mid point of the belt. This telescoping post includes a pivotal mirror at the distal end. This pivotal mirror allows for the visual inspection of the genital and rectum area of the user once the belt is attached around the waist of the individual.

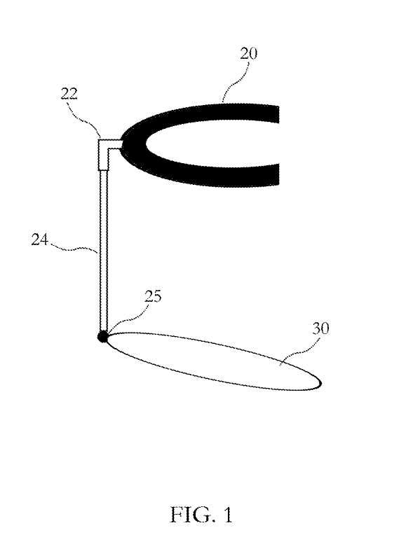

With respect to FIG. 1, a self-inspection device is depicted in accordance with the present invention. This self-inspection device includes a belt 20 that has an elbow attachment 22 extending from the midpoint of the belt 20. The elbow attachment 22 creates a 90-degree bend in a downward direction away from the belt 20. Extending from the elbow attachment 22 is a telescoping post 24. The telescoping post supports a mirror 30. The mirror 30 is positioned at the distal end of the telescoping post 24 with a pivoting connector 25. The pivoting connector 25 allows the user to pivot the mirror 30 at various angles during use. The belt 20 is attached around the user's waist and therefore the mirror 30 may be positioned between the legs of the user to allow for viewing of the genitals and rectum area of the user. The instant invention has been shown and described in what it considers to be the most practical and preferred embodiments. It is recognized, however, that departures may be made there from within the scope of the invention and that obvious modifications will occur to a person skilled in the art.

1. A self-inspection device comprising:
a belt, wherein said belt is adapted for placement around a user's waist;
an elbow attachment, where said elbow attachment is positioned at the center of the belt;
a telescoping post extending at a vertical angle from the elbow connector; and
a mirror, wherein said mirror is attached to the telescoping post.

2. The self-inspection device according to claim 1, further including a pivoting connector where pivoting connector connects the mirror to the telescoping post.

3. A method for self inspection of the genital and rectum areas of a user comprising:
wrapping a belt around a waist of a user;
positioning a mirror between the legs of the user; and
pivoting the mirror to view the genital and rectum areas of the user.
